(19) United States
(12) Patent Application Publication
(10) Pub. No.: US 2024/0287775 A1
(43) Pub. Date: Aug. 29, 2024

(54) Device and Method for Collecting and Cleaning Solid Substances

(71) Applicant: Stichting IMEC Nederland, Wageningen (NL)

(72) Inventors: Rogier Schoeman, Wageningen (NL); Neil Mc Sharry, Wageningen (NL)

(21) Appl. No.: 18/589,240

(22) Filed: Feb. 27, 2024

(30) Foreign Application Priority Data
Feb. 28, 2023 (EP) 23158915.1

(51) Int. Cl. E03C 1/264; B08B 1/50; B08B 3/04; B08B 3/06

(52) U.S. Cl. CPC E03C 1/264; B08B 1/50; B08B 3/045; B08B 3/06

(57) ABSTRACT

Example embodiments relate to devices and methods for collecting and cleaning solid substances. An example device is provided for cleaning dirt particles from a solid body. The device includes a driving shaft configured to rotate along a rotational direction. The device also includes a container arrangement that includes a first slotted container. Further, the device includes a second slotted container attached to the first slotted container and to the driving shaft via a fitting arrangement. The fitting arrangement is configured to simultaneously rotate the second slotted container within the first slotted container in a direction opposite to the rotational direction of the driving shaft.

### CROSS-REFERENCE TO RELATED APPLICATIONS

The present application is a non-provisional patent application claiming priority to European Patent Application No. EP23158915.1, filed Feb. 28, 2023, the contents of which are hereby incorporated by reference.

### FIELD OF THE DISCLOSURE

The disclosure relates to the collection and cleaning of solid substances, especially to provide a convenient means to retrieve and to clean ingestible sampling pills after excretion so that the contents of the capsule can be collected and tested.

### BACKGROUND

Generally, removing as much faeces as possible from the exterior of ingestible sampling pills, especially retrieved after excretion, is of paramount importance for the retrieval process and user experience. The process of cleaning the pill and the process of separating it from the faeces collectively reduce the risk of contamination of the sample during transport (e.g., to a testing facility). This process further improves the sanitation for those handling the pill and reduces any aversion to contact with faeces from the user retrieving it. For example, the device “CapsoRetrieve Collector Pan”, developed by CapsoVision Inc. (see https://capsovision.com/international/capsocam-accessories/), aims to solve the problem of the retrieval of sensing pills after excretion. The device facilitates a collection pan to be positioned in the toilet and after excretion, the cleaning of the retrieved pill is done by pouring water onto the pan (e.g., by flushing a toilet). A toilet flush may offer sufficient cleaning power to remove faeces on the higher end of the Bristol Stool Chart (i.e., stool which is runny, with a high-water content).

However, the cleaning power may not be sufficient to separate the pill from more solid faecal matter. Moreover, a pill embedded within a more viscous stool may not be liberated by a single flush and may require several flushes to achieve the preferred level of cleanliness. Multiple toilet flushes not only waste a considerable quantity of water but also time consuming and generally inefficient method.

### SUMMARY

The disclosure provides a device and a method for retrieving and cleaning solid substances (e.g., for retrieving ingestible sampling pills after excretion by collecting and cleaning the ingestible sampling pills to separate from the faeces) in a fast and efficient manner.

The object is solved by the features of the first independent claim for the device and by the features of the second independent claim for the method. The dependent claims contain further developments.

According to a first example, a device is provided for cleaning dirt particles from a solid body. The device comprises a driving shaft configured to rotate along a rotational direction, and a container arrangement comprising a first slotted container and a second slotted container attached to the first slotted container (e.g., positioned within the first slotted container) and further to the driving shaft via a fitting arrangement. In this regard, the fitting arrangement is configured to simultaneously rotate the second slotted container within the first slotted container in a direction opposite to the rotational direction of the driving shaft (e.g., based on the rotational movement of the driving shaft).

In some embodiments, the second slotted container is configured to hold the solid body. In other words, the solid body is to be collected or to be placed in the second slotted container (e.g., during the cleaning phase). The counter rotations of the second slotted container and the driving shaft may generate counter rotational forces (i.e., generating the turbulence and shear force) (e.g., around the solid body within the second slotted container).

The counter rotational force or shear force may be caused due to the viscous action of the fluid (e.g., water) around the solid body during the cleaning phase (e.g., as a result of the counter rotations of the second slotted container and the driving shaft). In this regard, the amount of shear force may be defined as the unit area amount of force that may act on the fluid parallel to the surface of the solid body.

Therefore, the combined cleaning force generated by the rotating parts (i.e., the centrifugal force generated through the rotation of the second slotted container and the turbulence and shear force generated through the opposing rotations of the second slotted container and the driving shaft) may provide a low impact method of cleaning to remove the dirt particles from the solid body in a fast and efficient manner, whereby reducing the risk of damage to the solid body surface.

In some embodiments, the driving shaft comprises a first member comprising a first end and a second end, and a second member attached to the first end of the first member via a further fitting arrangement. In this regard, the second end of the first member is configured to be detachably attached to the fitting arrangement. In another embodiment, the container arrangement can be placed at a suitable place such as at the narrow bottom section of a toilet and/or in a bucket, whereby the driving shaft may be detached during the collection phase (e.g., during excretion) and may be attached again during the cleaning and/or retrieving phase.

Furthermore, the second member is configured to move along the first member in a lateral movement direction. Moreover, the further fitting arrangement is configured to rotate the first member along the rotational direction based on the lateral movement of the second member.

In some embodiments, the linear to rotational motion conversion (i.e., the conversion of the linear motion of the second member into a one-way rotational motion of the first member) facilitates a comfortable and ease of cleaning operation (e.g., to rotate the second slotted container via the driving shaft). In another embodiment, the first member of the driving shaft further comprises a brush arrangement (e.g., at or near the second end). In an additional embodiment, the first member of the driving shaft (e.g., the brush arrangement) may comprise a plurality of bristles at or near the second end. In some embodiments, the brush arrangement may also contribute to the turbulent flow and may further facilitate a more aggressive cleaning to remove the dirt particles from the solid body.

In some embodiments, the first member of the driving shaft further comprises a plurality of lateral fins. The plurality of lateral fins may be at or near the second end. In some embodiments, the fins may further increase the turbulent flow, thereby increasing the low impact cleaning power.

In some embodiments, the first member of the driving shaft further comprises a first set of interlocking teeth at the first end. In an additional embodiment, the further fitting arrangement comprises a rotatable member comprising a second set of interlocking teeth configured to interlock with the first set of interlocking teeth of the first member. In a further embodiment, the rotatable member comprises longitudinal helical grooves.

Moreover, the second member of the driving shaft comprises a plurality of internal spheres configured to interlock with the longitudinal helical grooves of the rotatable member. In some embodiments, the linear motion of the second member can be converted into a one-way rotational motion of the first member in a simplified manner.

In some embodiments, the fitting arrangement comprises a first fastening member configured to attach the driving shaft to the fitting arrangement, whereby the first fastening member comprises a third set of interlocking teeth. In another embodiment, the fitting arrangement comprises a second fastening member comprising a fourth set of interlocking teeth configured to interlock with the third set of interlocking teeth of the first fastening member.

In a further embodiment, the second slotted container comprises a fifth set of interlocking teeth configured to interlock with the fourth set of interlocking teeth of the second fastening member. In some embodiments, the one-way rotational motion of the driving shaft can be converted into a one-way rotational motion of the second slotted container. In an additional embodiment, the rotational motion of the second slotted container may be in the opposite direction from the rotational motion of the driving shaft.

In some embodiments, the container arrangement further comprises a ball bearing assembly between the first slotted container and the second slotted container. This may facilitate the independent rotation of the second slotted container with respect to the first slotted container (e.g., within the first slotted container). In an additional embodiment, the first slotted container and the second slotted container each comprises a plurality of symmetrical or asymmetrical slots. The respective slots and the centrifugal force generated by the second slotted container may effectively carry away the dirt particles through the slots with ease.

In some embodiments, the first slotted container comprises a plurality of slots with a first slot dimension and the second slotted container comprises a plurality of slots with a second slot dimension, whereby the first slot dimension is greater than the second slot dimension. For example, the second slotted container may comprise slots with a slot dimension of about 7 mm×38 mm. The second slotted container may experience a greater exposure, which may further improve the cleaning process.

In some embodiments, the container arrangement is configured to be partially or fully submerged in a cleansing fluid (e.g., water). For example, the container arrangement may be placed at the narrow bottom section of a toilet or in an external water bucket. This may allow all the water from a toilet flush or in the bucket to be concentrated into one area, thereby facilitating more of the water to directly contact and clean the solid body.

In some embodiments, the concentration of water at the narrow bottom section of the toilet (e.g., combined with the added depth and the force of the oncoming water) may result in higher pressure and hence a faster flow of the water over the solid body, which may result in a greater cleaning force, thereby improving the water capacity for carrying away the dirt particles.

In some embodiments, the container arrangement, which may include the first slotted container and/or the second slotted container, comprises or is coated with a hydrophobic material or a superhydrophobic material such as polylactic acid (PLA), polyethylene (PE), high-density polyethylene (HDPE), polyetheretherketone (PEEK), poly-methyl methacrylate (PMMA), manganese-oxide polystyrene (MnO₂/PS), zinc-oxide polystyrene (ZnO/PS), precipitated calcium carbonate (PCC), and so on.

In some embodiments, the driving shaft, which may include the first member and/or the brush arrangement and/or the fins of the driving shaft, may comprise or be coated with a hydrophobic material or a superhydrophobic material.

In some embodiments, the solid body is an ingestible sampling pill. In another embodiment, the solid body may be one of the commercially available capsule endoscopy products. In an alternate embodiment, the solid body may correspond to any swallowed or anally lodged solid substances.

In some embodiments, a method is provided for cleaning dirt particles from a solid body. The method comprises the steps of providing a driving shaft being configured to rotate along a rotational direction, providing a container arrangement by attaching a second slotted container to a first slotted container and further to the driving shaft via a fitting arrangement, rotating the driving shaft along the rotational direction, and rotating the second slotted container simultaneously using the fitting arrangement within the first slotted container in a direction opposite to the rotational direction of the driving shaft.

In some embodiments, the method further comprises the steps of providing the driving shaft by attaching a second member to a first end of a first member via a further fitting arrangement and further by detachably attaching a second end of the first member to the fitting arrangement, moving the second member along the first member in a lateral movement direction, and rotating the first member using the further fitting arrangement along the rotational direction based on the lateral movement of the second member. In some embodiments, the method further comprises the steps of containing or holding the solid body by the second slotted container, and generating a counter rotational force or shear force at or around the solid body by simultaneously rotating the second slotted container in the direction opposite to the rotational direction of the driving shaft (i.e., by the opposite rotations of the driving shaft and the second slotted container). In some embodiments, the steps clean the dirt particles on the solid body.

In some embodiments, the method according to the second aspect corresponds to the device according to the first aspect and its implementation forms. Accordingly, the method of the second aspect may have corresponding implementation forms. In a further embodiment, the method of the second aspect achieves the same effects as the device of the first aspect and its respective implementation forms.

### BRIEF DESCRIPTION OF THE DRAWINGS

The above, as well as additional features will be better understood through the following illustrative and non-limiting detailed description, with reference to the appended drawings. In the drawings like reference numerals will be used for like elements unless stated otherwise.

FIG. 1 shows a device, according to example embodiments.

FIG. 2A shows a driving shaft, according to example embodiments.

FIG. 2B shows a driving shaft, according to example embodiments.

FIG. 2C shows a driving shaft, according to example embodiments.

FIG. 2D shows a driving shaft, according to example embodiments.

FIG. 3A shows a driving shaft, according to example embodiments.

FIG. 3B shows a driving shaft, according to example embodiments.

FIG. 4A shows a container arrangement, according to example embodiments.

FIG. 4B shows a container arrangement, according to example embodiments.

FIG. 4C shows a container arrangement, according to example embodiments.

FIG. 5A shows a fitting arrangement in detail, according to example embodiments.

FIG. 5B shows a container arrangement, according to example embodiments.

FIG. 6 shows a method, according to example embodiments.

### DETAILED DESCRIPTION

In cooperation with attached drawings, the technical contents and detailed description are described thereinafter according to example embodiments, being not used to limit the claimed scope. There are many different possibilities and the disclosure should not be construed as limited to the embodiments set forth herein; rather, these embodiments are provided for thoroughness and completeness, and fully convey the scope of the disclosure.

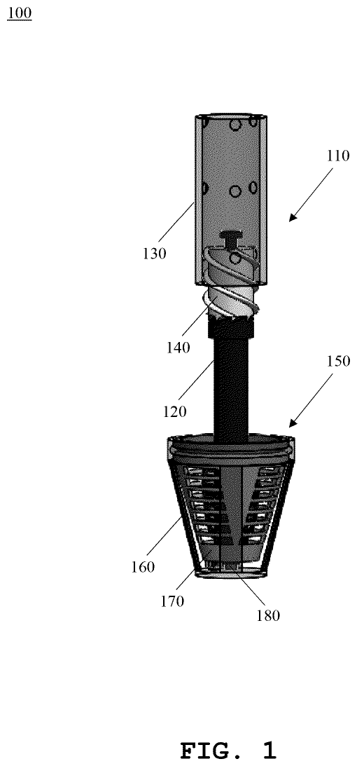

In FIG. 1, an example embodiment of the device 100 is illustrated. Particularly, the device 100 is provided for retrieving ingestible sampling pills after excretion (e.g., by collecting and cleaning the ingestible sampling pills to separate from the faeces).

The device 100 may comprise a driving shaft 110 that may further comprise a first member 120 or a shaft base, a second member 130 or a handle, and a fitting arrangement 140 or a rotating part. In this regard, the second member 130 may be moved along the first member 120 (e.g., over the fitting arrangement 140), whereby the fitting arrangement 140 may convert the linear motion of the second member 130 into one-way rotational motion, thereby rotating the first member 120 in a rotational direction.

The device 100 may further comprise a container arrangement 150 that may comprise a first slotted container 160 or a base container, a second slotted container 170 or a rotating container, and a fitting arrangement 180 or a gear arrangement. In some embodiments, the second slotted container 170 may be attached to the first slotted container 160 and further to the driving shaft 110. In some embodiments, the second slotted container 170 may be attached to the first member 120 of the driving shaft 110 via the fitting arrangement 180.

In some embodiments, the fitting arrangement 180 may cause the second slotted container 170 to rotate, which may occur by transferring the one-way rotational motion of the driving shaft 110 to the second slotted container 170. In some embodiments, the fitting arrangement 180 may rotate the second slotted container 170 within the first slotted container 160 in a direction opposite to the rotational direction of the driving shaft 110 (e.g., to the rotational direction of the first member 120 of the driving shaft 110).

The driving shaft 110 (e.g., the first member 120 of the driving shaft 110) may be detachably attached to the fitting arrangement 180 within the container arrangement 150. In other words, the driving shaft 110 and the container arrangement 150 may be separable.

The container arrangement 150 may comprise a conical shape, such as a flat-conical shape flattened at the bottom end of the container arrangement 150. In another embodiment, the container arrangement 150 may comprise a tapered-cylindrical shape. In an alternative embodiment, the container arrangement 150 may comprise a cylindrical shape. The second slotted container 170 may have a smaller dimension compared to the first slotted container 160 in order to facilitate the rotation of the second slotted container 170 within the first slotted container 160.

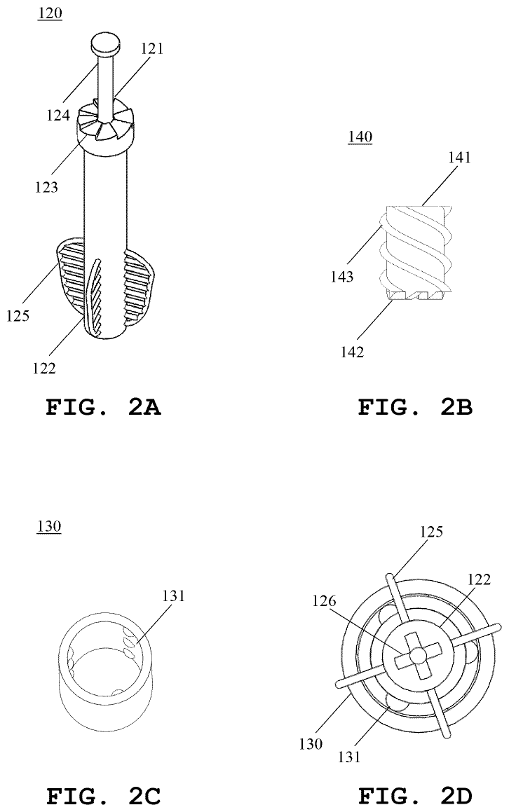

In FIGS. 2A-2D, an example embodiment of the driving shaft 110 is illustrated. In particular, FIG. 2A shows the first member 120 of the driving shaft 110 in detail. The first member 120 may comprise a first end 121 and a second end 122. The first end 121 may comprise a set of interlocking teeth 123. The first end 121 may further comprise a thinned region 124 (e.g., to position or to affix the fitting arrangement 140 at the first end 121 of the first member 120). As such, the first member 120 may comprise a solid cylindrical shape with the thinned region 124 and the set of interlocking teeth 123 facilitated at the first end 121. Furthermore, the second end 122 may comprise a plurality of fins or fan blades 125, and may include a plurality of fins laterally affixed along the second end 122 of the first member 120. The fins 125 may comprise or be slotted fins and/or solid fins.

The fins 125 may comprise a rectangular shape, a swept shape, a tapered swept shape, a clipped delta shape, a trapezoidal shape, an elliptical shape, or the like. The fins 125 may be formed integrally with respect to the first member 120 (i.e., comprising a material homogeneous to the material of the first member 120).

Alternatively, the fins 125 may comprise a heterogeneous material and may be affixed to the first member 120 at the second end 122 via glue or screws or other fasteners. The second end 122 may further comprise recesses 126, which is shown in FIG. 2D, in order to attach the first member 120 with the fitting arrangement 180 of the container arrangement 150.

FIG. 2B shows the fitting arrangement 140 of the driving shaft 110 in detail. The fitting arrangement 140 may comprise or be a rotatable member 141 comprising a hollowed-cylindrical shape with a set of interlocking teeth 142, wherein the interlocking teeth 142 may be formed at one end of the rotatable member 141, and longitudinal helical grooves 143, wherein the longitudinal helical grooves 143 may be formed at the outer surface of the rotatable member 141.

For example, the set of interlocking teeth 142 may be formed to interlock with the set of interlocking teeth 123 at the first end 121 of the first member 120. Furthermore, the hollowed inner section of the rotatable member 141 may be formed to match the thinned region 124 at the first end 121 of the first member 120 (e.g., to affix the rotatable member 141 at the first end 121 of the first member 120).

The fitting arrangement 140, and/or the rotatable member 141, may be formed integrally with respect to the first member 120 (i.e., comprising a material homogeneous to the material of the first member 120). Alternatively, the fitting arrangement 140 or the rotatable member 141 may comprise a homogeneous or a heterogeneous material and may be affixed to the first member 120 at the first end 121 via glue or screws or other fasteners.

FIG. 2C shows the second member 130 of the driving shaft 110 in detail. The second member 130 may comprise a hollowed-cylindrical shape with a plurality of internal spheres 131, wherein the internal spheres 131 may be formed at the inner surface of the second member 130.

The hollowed-cylindrical shape of the second member 130 may be formed so as to encompass the fitting arrangement 140 and/or the rotatable member 141, at the first end 121 of the first member 120 while moving along the first member 120 over the rotatable member 141 in the lateral movement direction. The plurality of internal spheres 131 of the second member 130 may be formed to interlock with the longitudinal helical grooves 143 of the rotatable member 141. In some embodiments, the plurality of internal spheres 131 of the second member 130 may be formed to interlock with the longitudinal helical grooves 143 of the rotatable member 141 while moving in the lateral movement direction.

FIG. 2D shows a bottom view of the driving shaft 110. In some embodiments, the first member 120 (e.g., the bottom edge at the second end 122) may comprise recesses 126 (e.g., cross-shaped recesses as shown herein) in order to attach the first member 120 with the fitting arrangement 180 of the container arrangement 150 in a detachable manner.

Therefore, the second member 130 may be freely moved in the lateral movement direction over the rotatable member 141 along the first member 120. The interlocking between the plurality of internal spheres 131 of the second member 130 and the longitudinal helical grooves 143 of the rotatable member 141 may cause the rotatable member 141 to rotate in the rotational direction, thereby translating or converting the lateral movement of the second member 130 into one-way rotational movement of the rotatable member 141.

Furthermore, the interlocking between the set of interlocking teeth 123 of the first member 120 and the set of interlocking teeth 142 of the rotatable member 141 may cause the first member 120 to rotate in the rotational direction, thereby transferring the one-way rotational movement of the rotatable member 141 to the first member 120.

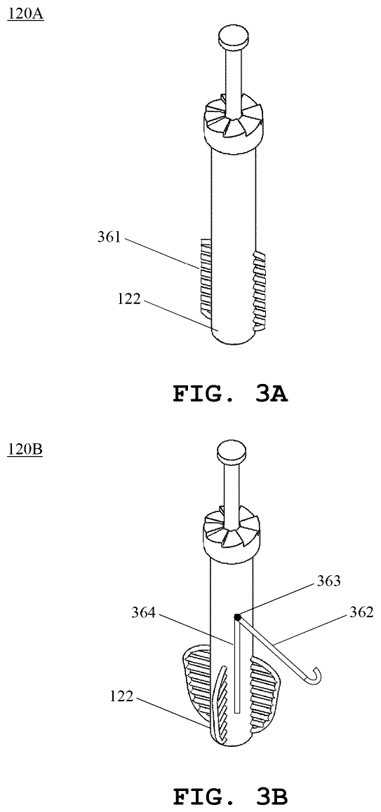

FIG. 3A shows an example embodiment of the driving shaft 110. In particular, an embodiment of the first member 120A of the driving shaft 110 is illustrated herein. The first member 120A differs from the first member 120 in that the first member 120A may comprise a brush arrangement 361 at the second end 122 of the first member 120A (e.g., in addition to or instead of the plurality of fins 125). The brush arrangement 361 may comprise a plurality of bristles that include or are made of synthetic fiber (e.g., polyamide, polyester, polypropylene, and so on). FIG. 3B shows an example embodiment of the driving shaft 110. In particular, an embodiment of the first member 120B of the driving shaft 110 is illustrated herein. The first member 120B differs from the first member 120 in that the first member 120B may comprise a hook arrangement 362 in order to facilitate the lifting of the container arrangement 150 for retrieving the pills after the cleaning phase and/or to re-position the container arrangement 150 to a different location and/or setup. In this regard, the hook arrangement 362 may comprise a hook with an elongated end, which may be attached to the first member 120B via screws or springs 363. The first member 120B may further comprise a shallow groove 364, wherein the shallow groove 364 may be at the outer surface, in order to fully accommodate and thereby encompassing the hook arrangement 362.

For example, during the cleaning phase (i.e., when the first member 120B is rotated) the hook arrangement 362 may reside within the shallow groove 364. Furthermore, after the cleaning phase (i.e., when the first member 120B is standstill) the hook arrangement 362 may be ejected or be sprung from the shallow groove 364 (e.g., either manually or via the spring 363).

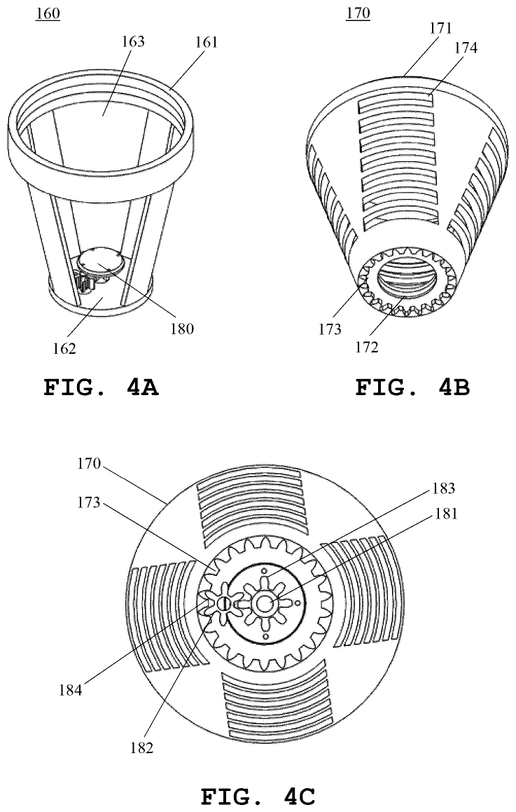

FIGS. 4A-4C show an embodiment of the container arrangement 150. In particular, FIG. 4A shows the first slotted container 160 of the container arrangement 150 in detail. The first slotted container 160 may comprise or be a hollowed-tapered cylindrical container comprising a hollowed top part 161 and a solid bottom part or base 162. The first slotted container 160 may further comprise a plurality of slots 163 (e.g., comprising slots tapered from the top part 161 to the bottom part 162). The first slotted container 160 may further comprise the fitting arrangement 180 (e.g, at the solid bottom part 162). FIG. 4B shows the second slotted container 170 of the container arrangement 150 in detail. The second slotted container 170 may comprise or be a hollowed-tapered cylindrical container comprising a hollowed top part 171 and a hollowed bottom part or base 172. The second slotted container 170 may further comprise a set of interlocking teeth 173 (e.g., at the bottom part 172).

The second slotted container 170 may further comprise a plurality of slots 174 (e.g., comprising slots formed along the tapered surface from the top part 171 to the bottom part 172). For example, each of the plurality of slots 174 may comprise a slot dimension of about 7 mm×38 mm.

FIG. 4C shows the fitting arrangement 180 of the container arrangement 150. The fitting arrangement 180 may comprise a first fastening member or gear 181 comprising a set of interlocking teeth 183. The fitting arrangement 180 may further comprise a second fastening member or gear 182 comprising a set of interlocking teeth 184.

In some embodiments, the set of interlocking teeth 184 of the second fastening member 182 may be formed to interlock with the set of interlocking teeth 183 of the first fastening member 181. Furthermore, the set of interlocking teeth 184 of the second fastening member 182 may be formed to interlock with the set of interlocking teeth 173 of the second slotted container 170 (e.g., at the bottom part 172).

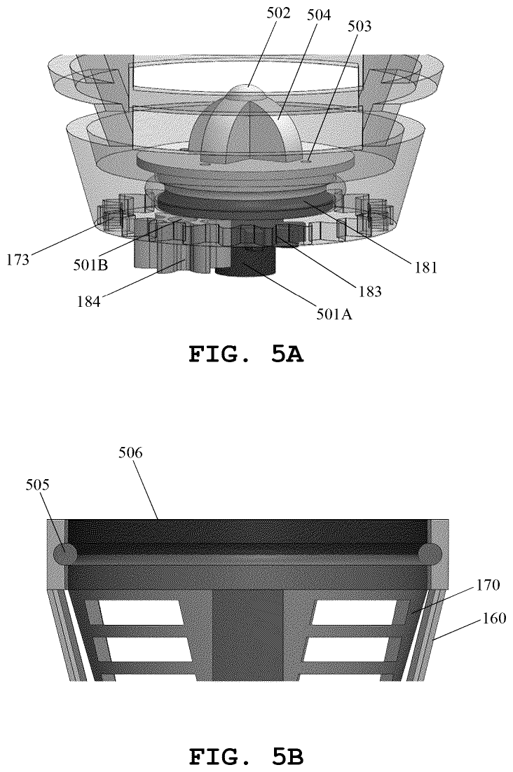

FIG. 5A further shows the fitting arrangement 180 of the container arrangement 150 in detail. The fitting arrangement 180 may further comprise a first base 501A and a second base 501B. The first base 501A and the second base 501B may be fixed at the solid bottom part 162 of the first slotted container 160.

In this regard, the first base 501A may be formed to affix the first fastening member 181 in a rotatable manner (i.e., to facilitate a free rotation of the first fastening member 181 at the first base 501A). Furthermore, the second base 501B may be formed to affix the second fastening member 182 in a rotatable manner (i.e., to facilitate a free rotation of the second fastening member 182 at the second base 501B).

Moreover, the fitting arrangement 180 (e.g., the first fastening member 181) may comprise a third fastening member 502. In some embodiments, the third fastening member 502 may be attached to the first fastening member 181. The third fastening member 502 may comprise a flat base 504 and a set of interlocking slots 503. The flat base 504 may be formed to affix the third fastening member 502 on top of the first fastening member 181 (e.g., via glue or screws).

In another embodiment, the third fastening member 502 may be integrally formed with the first fastening member 181. In some embodiments, the set of interlocking slots 503 may be formed to interlock with the recesses 126 of the first member 120 of the driving shaft 110. In some embodiments, the set of interlocking slots 503 may be formed to interlock with the recesses 126 of the first member 120 of the driving shaft 110 at the second end 122 of the first member 120 of the driving shaft 110.

In some embodiments, the hollowed bottom part 172 of the second slotted container 170 may comprise an inner-diameter such that the third fastening member 502 may be passed through and be able to attach the driving shaft 110 accordingly. In some embodiments, the rotational movement of the driving shaft 110, which may include the first member 120 of the driving shaft 110, along the rotational direction may be transferred to the first fastening member 181 via the third fastening member 502. In an additional embodiment, the interlocking between the set of interlocking teeth 183 of the first fastening member 181 and the set of interlocking teeth 184 of the second fastening member 182 may cause the second fastening member 182 to rotate in a direction opposite to the rotational direction of the first fastening member 181.

In a further embodiment, the set of interlocking teeth 184 of the second fastening member 182 and the set of interlocking teeth 173 of the second slotted container 170 may cause the second slotted container 170 to rotate in the rotational direction of the second fastening member 182.

The fitting arrangement 180 of the container arrangement 150 may translate the rotational movement of the driving shaft 110 to the container arrangement 150 (e.g., to rotate the second slotted container 170 within the first slotted container 160 in the direction opposite to the rotational direction of the driving shaft 110).

FIG. 5B shows an embodiment of the container arrangement 150. The container arrangement 150 may comprise a ball-bearing arrangement or assembly 505 between the first slotted container 160 and the second slotted container 170. For example, the hollowed top part 161 of the first slotted container 160 and the hollowed top part 171 of the second slotted container 170 may be formed (e.g., with grooves or channels) to comprise the ball-bearing 50 between the respective top parts.

In a further embodiment, the container arrangement 150 may further comprise a cap 506 to encompass the respective top parts of the container arrangement 150 as well as to cover the ball-bearing 50, whereby allowing the free rotation of the second slotted container 170 within the first slotted container 160. The cap 506 may additionally provide protection against faeces and/or toilet paper from interfering with the ball-bearing 50.

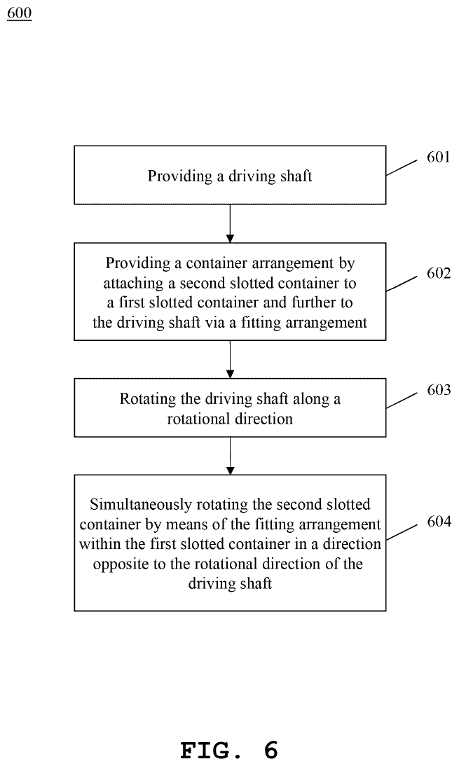

In an additional embodiment, the container arrangement 150 may comprise a further ball-bearing (not shown) between the hollowed bottom part 172 of the second slotted container 170 and the first fastening member 181/the third fastening member 502 (e.g., to enhance the free rotation of the second slotted container 170). In FIG. 6, an example embodiment of a method 600 is illustrated. In a first step 601, a driving shaft is provided, the driving shaft being configured to rotate along a rotational direction. In a second step 602, a container arrangement is provided by attaching a second slotted container to a first slotted container and further to the driving shaft via a fitting arrangement.

In some embodiments, during a third step 603, the driving shaft is rotated along the rotational direction. In some embodiments, during a fourth step 604, the second slotted container is simultaneously rotated using the fitting arrangement within the first slotted container in a direction opposite to the rotational direction of the driving shaft.

The disclosure utilizes a combination of turbulence and shear force, centrifugal force, and mechanical process to clean sensing pills (e.g., retrieved after excretion). The turbulence and shear may be generated through the opposing rotations of the rotating container and the driving shaft and optionally of the fins and/or the brushes attached to the driving shaft. Use of the turbulence and shear force may provide a low impact way of cleaning, thereby reducing the risk of damage to the pill.

Moreover, the brush arrangement, which also may contribute to the turbulent flow, may provide a slightly more aggressive cleaning component to aid in the breakup of larger or tougher pieces of faeces while posing minimum risk to the pill's integrity. The spinning motion of the rotating container may impart a centrifugal force on the faecal fragments contained, which may in turn pull them to the sides of the rotating container and through the slots. The slots, including the slots of the rotating container, may be sized so as not to allow the pill itself to escape but only to remove the pieces of faeces.

It is to be noted that human faeces may experience a thinning behavior with shear rates as low 1 s⁻¹. Furthermore, at shear rates of 10 s⁻¹ for seconds, the viscosity of the faeces may decrease to 0.5% of its original value. The example embodiments may generate a shear rate of at least one to two orders of magnitude higher than the above value.

What is claimed is:

1. A device for cleaning dirt particles from a solid body comprising:
a driving shaft configured to rotate along a rotational direction;
a container arrangement comprising a first slotted container; and
a second slotted container attached to:
the first slotted container; and
the driving shaft via a fitting arrangement,
wherein the fitting arrangement is configured to simultaneously rotate the second slotted container within the first slotted container in a direction opposite to the rotational direction of the driving shaft.

2. The device according to claim 1, wherein the driving shaft comprises:
a first member comprising a first end and a second end; and
a second member attached to the first end of the first member via a further fitting arrangement,
wherein the second end of the first member is configured to be detachably attached to the fitting arrangement,
wherein the second member is configured to move along the first member in a lateral movement direction, and
wherein the further fitting arrangement is configured to rotate the first member along the rotational direction based on the lateral movement of the second member.

3. The device according to claim 2, wherein the first member of the driving shaft further comprises a brush arrangement.

4. The device according to claim 3, wherein the brush arrangement is positioned at or near the second end of the first member.

5. The device according to claim 2, wherein the first member of the driving shaft further comprises a plurality of lateral fins at or near the second end.

6. The device according to claim 2,
wherein the first member further comprises a first set of interlocking teeth at the first end,
wherein the further fitting arrangement comprises a rotatable member, and
wherein the rotatable member comprises a second set of interlocking teeth configured to interlock with the first set of interlocking teeth of the first member.

7. The device according to claim 6,
wherein the rotatable member further comprises longitudinal helical grooves, and
wherein the second member comprises a plurality of internal spheres configured to interlock with the longitudinal helical grooves of the rotatable member.

8. The device according to claim 1, wherein the fitting arrangement comprises:
a first fastening member configured to attach the driving shaft to the fitting arrangement, wherein the first fastening member comprises a third set of interlocking teeth; and
a second fastening member comprising a fourth set of interlocking teeth configured to interlock with the third set of interlocking teeth of the first fastening member.

9. The device according to claim 8, wherein the second slotted container comprises a fifth set of interlocking teeth configured to interlock with the fourth set of interlocking teeth of the second fastening member.

10. The device according to claim 1, wherein the container arrangement further comprises a ball bearing assembly between the first slotted container and the second slotted container.

11. The device according to claim 1, wherein the first slotted container and the second slotted container each comprises a plurality of symmetrical or asymmetrical slots.

12. The device according to claim 1,
wherein the first slotted container comprises a plurality of slots with a first slot dimension and the second slotted container comprises a plurality of slots with a second slot dimension, and
wherein the first slot dimension is greater than the second slot dimension.

13. The device according to claim 1, wherein the container arrangement is configured to be partially or fully submerged in a cleansing fluid.

14. The device according to claim 1, wherein the solid body is an ingestible sampling pill.

15. The device according to claim 1, wherein the container arrangement comprises or is coated with a hydrophobic material or a superhydrophobic material.

16. The device according to claim 15, wherein the first slotted container or the second slotted container comprises or is coated with the hydrophobic material or the superhydrophobic material.

17. A method for cleaning dirt particles from a solid body comprising:
providing a driving shaft configured to rotate along a rotational direction;
providing a container arrangement by attaching a second slotted container to a first slotted container and further to the driving shaft via a fitting arrangement;
rotating the driving shaft along the rotational direction; and
rotating the second slotted container simultaneously using the fitting arrangement within the first slotted container in a direction opposite to the rotational direction of the driving shaft.

18. The method according to claim 17, further comprising:
providing the driving shaft by:
attaching a second member to a first end of a first member via a further fitting arrangement; and
detachably attaching a second end of the first member to the fitting arrangement;
moving the second member along the first member in a lateral movement direction; and
rotating the first member using the further fitting arrangement along the rotational direction based on the lateral movement of the second member.

19. The method according to claim 17, further comprising:
containing the solid body by the second slotted container; and
generating a counter rotational force at or around the solid body by simultaneously rotating the second slotted container in the direction opposite to the rotational direction of the driving shaft.

20. The method according to claim 19, wherein generating the counter rotational force at or around the solid body by simultaneously rotating the second slotted container in the direction opposite to the rotational direction of the driving shaft cleans the dirt particles on the solid body.
